(19) United States
(12) Patent Application Publication
(10) Pub. No.: US 2006/0110409 A1
(43) Pub. Date: May 25, 2006

(54) Targeted agents for nerve regeneration

(76) Inventors: Clifford Charles Shone, Salisbury (GB); John Mark Sutton, Salisbury (GB)

Correspondence Address: EVAN LAW GROUP LLC, 566 WEST ADAMS, SUITE 350, CHICAGO, IL, 60661, US

(21) Appl. No.: 10/521,401

(22) PCT Filed: Jul. 15, 2003
(86) PCT No.: PCT/GB03/03082

§ 371 Date: Sep. 12, 2005

(30) Foreign Application Priority Data
Jul. 19, 2002 (GB) 0216865.6

(51) Int. Cl. A61K 39/08; C12N 9/48

(52) U.S. Cl. 424/239.1; 435/212

(57) ABSTRACT

A conjugate, for delivery of a therapeutic agent to a neuronal cell, comprises the therapeutic agent, a binding domain that binds to the neuronal cell, and a translocation domain that translocates the therapeutic agent into the neuronal cell, wherein the binding domain is H_{C} of botulinum C₁ toxin or is based thereon.

The present invention relates to delivery of agents to neuronal cells, especially agents that promote nerve regeneration, to constructs for delivering the agents, to associated use of the agents and constructs and to manufacture thereof.

There are presently few effective treatments for major disorders of the central nervous system. Such disorders include neurodegenerative diseases, stroke, epilepsy, brain tumours, infections and HIV encephalopathy, and sufferers of these diseases far outnumber the morbidity of cancer and heart disease. As our understanding of brain pharmacology increases and the underlying pathologies of diseases are elucidated, potential therapeutic strategies become apparent. All these treatments, however, face the formidable problem of efficient delivery of therapeutics to the various neuronal cell populations involved. Vectors which can effect efficient delivery to neuronal cells are thus required for a broad range of therapeutic substances, including drugs, enzymes, growth factors, therapeutic peptides and genes.

A major problem in the use of such therapies is the delivery of useful concentrations of the active agent to the site of trauma. Specific neuronal vectors could therefore play an important role in targeting such compounds to neuronal cells.

Suitable neuronal cell-specific targeting ligands are therefore required for a broad range of gene vectors to enable effective treatments for neuronal diseases to be developed.

The clostridial neurotoxins are protein toxins produced by various species of the genus Clostridium, most importantly C. tetani and several strains of C. botulinum. There are at present eight different classes of the neurotoxins known: tetanus toxin and botulinum neurotoxin in its serotypes A, B, C₁, D, E, F and G, and they all share similar structures and modes of action. The clostridial neurotoxins are synthesized by the bacterium as a single polypeptide that is modified post-translationally to form two polypeptide chains joined together by a disulphide bond. The two chains are termed the heavy chain (H), and the light chain (LC). The clostridial neurotoxins are highly selective for neuronal cells, and bind with high affinity thereto.

The botulinum neurotoxins are a sub-family of clostridial neurotoxins whose primary site of action is the neuromuscular junction where they block the release of the transmitter acetylcholine. Tetanus toxin is structurally very similar to botulinum neurotoxins but its primary site of action is the central nervous system where it blocks the release of inhibitory neurotransmitters from central synapses (Renshaw cells).

The neuronal cell targeting of tetanus and botulinum neurotoxins is highly specific and is considered to be a receptor mediated event following which the toxins become internalised and subsequently traffic to the appropriate intracellular compartment where they effect their endopeptidase activity.

It is possible to provide functional definitions of the heavy chain domains within the clostridial neurotoxin molecules, as follows
clostridial neurotoxin heavy chain H_{C} domain
a portion of the heavy chain which is responsible for binding of the native holotoxin to cell surface receptor(s) involved in the intoxicating action of clostridial toxin prior to internalisation of the toxin into the cell.
clostridial neurotoxin heavy chain H_{N} domain
a portion of the heavy chain which enables translocation of that portion of the neurotoxin molecule such that a functional expression of light chain activity occurs within a target cell.
the domain responsible for translocation of the endopeptidase activity, following binding of neurotoxin to its specific cell surface receptor via the binding domain, into the target cell.
the domain responsible for formation of ion-permeable pores in lipid membranes under conditions of low pH.

Tetanus and the botulinum neurotoxins from most of the seven serotypes, together with their derived heavy chains, have been shown to bind a wide variety of neuronal cell types with high affinities in the nM range, e.g. botulinum type B neurotoxin (Evans et al. (1986) Eur. J. Biochem. 154, 409-416).

It is known to use H_{C} domains from botulinum toxins A and B to provide specific targeting of therapeutic agents to neuronal cells.

However, a problem that has been identified with these targeted constructs is that when the H_{C} domain is made recombinantly, the affinity and specificity for neuronal cells is significantly reduced, and this can be up to the point where there is no effective targeting of neuronal cells. To date, while attempts have been made to modify the H_{C} regions from A and B toxins to overcome this difficulty there has been no success.

Separately, it is known to use an adenoviral vector to deliver to a neuronal cell a gene that codes for C3 exoenzyme to promote central nervous system axon regeneration. This approach, however, has the disadvantage of the risk of viral proliferation in the patient receiving the treatment. As a result, it would be unlikely ever to receive approval for human use.

An object of the present invention is to provide agents that are targeted to neuronal cells and which can be used to deliver therapeutic agents thereto. An object of specific embodiments of the invention is to provide targeted delivery to neuronal cells of agents that can promote nerve regeneration. A further object is to overcome or at least improve upon the problems and disadvantages identified in the prior art.

The invention provides in its various aspects, targeted delivery of therapeutic agents, e.g. an inhibitor of Rho function, to neural cells, to promote nerve regeneration; and constructs for targeted delivery of therapeutic agents to neuronal cells, using an H_{C} domain from botulinum C₁ toxin, especially one made recombinantly.

Accordingly, a first aspect of the invention provides a composition, for delivery of a therapeutic agent to a neuronal cell, comprising
the therapeutic agent, and
a neuronal cell targeting component, which component comprises a H_{C} domain of botulinum C₁ toxin, or a fragment, variant, or derivative thereof which retains the function of the native H_{C} domain.

Thus according to the invention, an H_{C} portion from botulinum toxin C₁ is made recombinantly and used to provide targeting to neuronal cells of therapeutic agents. The H_{C} portion retains its binding affinity for neuronal cells, in contrast to the corresponding chains from A and B toxins.

Compositions of the invention suitably further comprise a translation domain, for translocation of the therapeutic agent into a target cell. The neuronal cell targeting agent may be linked, e.g. covalently, using linkages which may include one or more spacer regions, to a translocation domain to effect transport of the therapeutic agent into the cytosol. Examples of translocation domains derived from clostridial neurotoxins are as follows

| | |
|---|---|
| Botulinum type A neurotoxin | amino acid residues (449-871) |
| Botulinum type B neurotoxin | amino acid residues (441-858) |
| Botulinum type C neurotoxin | amino acid residues (442-866) |
| Botulinum type D neurotoxin | amino acid residues (446-862) |
| Botulinum type E neurotoxin | amino acid residues (423-845) |
| Botulinum type F neurotoxin | amino acid residues (440-864) |
| Botulinum type G neurotoxin | amino acid residues (442-863) |
| Tetanus neurotoxin | amino acid residues (458-879) |

Other clostridial sources of translocation domains include—C. butylicum, and C. argentinense, and for the genetic basis of toxin production in Clostridium botulinum and C. tetani, see Henderson et al (1997) in The Clostridia: Molecular Biology and Pathogenesis, Academic press.

In addition to the above translocation domains derived from clostridial sources, other non-clostridial sources may be employed in a construct according to the present invention. These include, for example, diphtheria toxin [London, E. (1992) Biochem. Biophys. Acta., 1112, pp. 25-51], Pseudomonas exotoxin A [Prior et al (1992) Biochem., 31, pp. 3555-3559], influenza virus haemagglutinin fusogenic peptides [Wagner et al (1992) PNAS, 89, pp. 7934-7938], and amphiphilic peptides [Murata et al (1992) Biochem., 31, pp. 1986-1992].

In preferred embodiments of the invention, a translocation domain is selected from botulinum toxin C₁ translocation domain and functional fragments and derivatives thereof, and diphtheria toxin translocation domain and functional fragments and derivatives thereof.

In use, the domains of a construct according to the present invention are associated with each other. In one embodiment, two or more of the domains may be joined together either directly (eg. by a covalent linkage), or via a linker molecule. Conjugation techniques suitable for use in the present invention have been well documented
Chemistry of protein conjugation and cross-linking. Edited by Wong, S. S. 1993, CRC Press Inc., Florida; and
Bioconjugate techniques, Edited by Hermanson, G. T. 1996, Academic Press, London, UK.

Direct linkage of two or more domains is now described with reference to embodiments employing clostridial neurotoxins or fragments thereof and to the following nomenclature of clostridial neurotoxin domains, namely Domain B (contains the neuronal cell targeting domain), Domain T (contains the translocation domain) and Domain E (contains the therapeutic agent), although no limitation thereto is intended.

In one embodiment of the present invention, Domains E and T may be mixed together in equimolar quantities under reducing conditions and covalently coupled by repeated dialysis (eg. at 4° C., with agitation), into physiological salt solution in the absence of reducing agents. At this stage, in contrast to Example 6 of WO94/21300, the E-T complex is not blocked by iodoacetamide, therefore any remaining free —SH groups are retained. Domain B is then modified, for example, by derivatisation with the coupling agent SPDP followed by subsequent reduction. In this reaction, SPDP does not remain attached as a spacer molecule to Domain B, but simply increases the efficiency of this reduction reaction. Reduced Domain B and the E-T complex may then be mixed under non-reducing conditions (eg. at 4° C.) to form a disulphide-linked E-T-B construct.

In another embodiment, a coupled E-T complex may be prepared according to Example 6 of WO94/21300, including the addition of iodoacetamide to block free sulphydryl groups. However, the E-T complex is not further derivatised, and the remaining chemistry makes use of the free amino (—NH₂) groups on amino acid side chains (eg. lysine, and arginine amino acids).

Domain B may be derivatised using carbodiimide chemistry (eg. using EDAC) to activate carboxyl groups on amino acid side chains (eg. glutamate, and aspartate amino acids), and the E-T complex mixed with the derivatised Domain B to result in a covalently coupled (amide bond) E-T-B construct.

Suitable methodology for the creation of such a construct is, for example, as follows

Domain B was dialysed into MES buffer (0.1 M MES, 0.1 M sodium chloride, pH 5.0) to a final concentration of 0.5 mg/ml. EDAC (1-ethyl-3-[3-dimethylaminopropyl] carbodiimide hydrochloride) was added to final concentrations of 0.2 mg/ml and reacted for 30 min at room temperature. Excess EDAC was removed by desalting over a MES buffer equilibrated PD-10 column (Pharmacia). The derivatised domain B was concentrated (to >2 mg/ml) using Millipore Biomax 10 concentrators. The E-T complex (1 mg/ml) was mixed for 16 hours at 4° C., and the E-T-B complex purified by size-exclusion chromatography over a Superose 12 HR10/30 column (Pharmacia) to remove unreacted Domain B (column buffer: 50 mM sodium phosphate pH6.5+20 mM NaCl).

As an alternative to direct covalent linkage of the various Domains of a construct/composition according to the present invention, suitable linker molecules may be employed. The term linker molecule is used synonymously with spacer molecule. Spacer technology was readily available prior to the present application.

For example, one particular coupling agent (SPDP) is described in Example 6 of WO94/21300 (see lines 3-5 on page 16). In Example 6, SPDP is linked to an E-T complex, thereby providing an E-T complex including a linker molecule. This complex is then reacted with a Domain B, which becomes attached to the E-T complex via the linker molecule. In this method, SPDP results in a spacing region of approximately 6.8 Angstroms between different Domains of the construct of the present invention.

A variant of SPDP known as LC-SPDP is identical in all respects to SPDP but for an increased chain length. LC-SPDP may be used to covalently link two Domains of the construct of the present invention resulting in a 15.6 Angstrom spacing between these Domains.

Examples of spacer molecules include, but are not limited to

| | |
|---|---|
| (GGGGS)₂, elbow | [see Anand et al. (1991) J. Biol. |
| regions of Fab | Chem. 266, 21874-9]; |
| (GGGGS)₃ | [see Brinkmann et al. (1991) Proc. |
| | Natl. Acad. Sci. 88, 8616-20]; |
| the interdomain | [see Takkinen et al. (1991) Protein |
| linker of cellulase | Eng, 4, 837-841]; |
| PPPIEGR | [see Kim (1993) Protein Science, 2, |
| | 348-356]; |
| Collagen-like spacer | [see Rock (1992) Protein |
| | Engineering, vol 5, No 6, |
| | pp583-591]; and |
| Trypsin-sensitive | [see O'Hare (1990) FEBS, vol 273, |
| diphtheria | No 1, 2, pp 200-204]. |
| toxin peptide | |

In a further embodiment of the present invention, a construct having the structure E-X-T-X-B, where “X” is a spacer molecule between each domain, may be prepared, for example, as follows

Domain E is derivatised with SPDP, but not subsequently reduced. This results in an SPDP-derivatised Domain E. Domain T is similarly prepared, but subsequently reduced with 10 mM dithiothreitol (DTT). The 10 mM DTT present in the Domain T preparation, following elution from the QAE column (see Example 6 in WO94/21300), is removed by passage of Domain T through a sephadex G-25 column equilibrated in PBS. Domain T free of reducing agent is then mixed with the SPDP-derivatised Domain E, with agitation at 4° C. for 16 hours. E-T complex is isolated from free Domain E and from free Domain T by size-exclusion chromatography (Sephadex G-150). Whereafter, the same procedure can be followed as described in Example 6 of WO94/21300 for rederivatisation of the E-T complex with SPDP, and subsequent coupling thereof to the free sulphydryl on Domain B to form.

In a construct of the invention, the translocation domain (Domain T) is not limited, and could be any translocation domain. Translocation domains can frequently be identified by the property of being able to form measurable pores in lipid membranes at low pH (Shone et al. Eur J. Biochem. 167, 175-180).

The therapeutic agent could, similarly, be any suitable agent and reference to “therapeutic substance” or “therapeutic agent” is a reference to any substance, agent or mixture thereof, which, if delivered by or in the composition of the invention, is beneficial in the treatment of disease. Examples of these include drugs, growth factors, enzymes, and DNA packaged in various forms (e.g. modified viruses, cationic liposomes, and condensed DNA).

For promoting nerve regeneration, any Rho inhibitor can be used, especially a C3 exoenzyme. The C3 family of exoenzymes is defined as a family of proteins of 20-30 kDa with a basic isolectric point usually greater than 9. The enzymes are ADP-ribosyltransferases, which modify small GTPases, usually of the Rho family, by the addition of ADP-ribose. This usually results in the inactivation of the GTPases.

Members of the C3 exoenzyme family described to date include at least 2 isoforms from Clostridium botulinum, an isoform from C. limosum, an isoform from Bacillus cereus, and 3 isoforms from Staphylococcus aureus. C3 isoforms from C. acetobutylicum, Streptococcus pyogenes and Listeria monocytogenes are disclosed for the first time herein and represent further embodiments of the invention.

Enzymes having C3 activity can be identified, for example, using the assay described in Example 1.

C3-like toxins can be further defined by the presence of motifs covering two elements of the active site such that all examples carry the consensus S S/T S/T Hyd X35-40 Q/E X E Hyd Hyd Hyd where Hyd represents any hydrophobic residue (usually I, L, V, G or A) and X represents any amino acid. The motif usually has an aromatic residue in one of the two preceding positions. The S S/T S/T motif is a key determinant of substrate specificity. Hence, a C3 enzyme used in the present invention is preferably one that includes these motifs.

Further aspects of the invention provide a polypeptide construct comprising an inhibitor of a member of the Rho family of GTPases, for use in neuronal cell therapy; and use of an inhibitor of a member of the Rho family of GTPases, for use in manufacture of a medicament for neuronal cell therapy, the inhibitor in both cases suitably being or comprising a C3 enzyme.

In use, advantages of constructs of the invention include one or more of
targeted to the neuron, therefore no cytotoxic effect on non-neuronal cells,
the enzymic action of C3, in which one C3 molecule can inactivate many target (Rho) molecules, provides efficient inhibition which is more effective than conventional (drug-like) inhibitors which act by binding to their target in a one-to-one ratio,
much smaller molecular size compared to virus vectors, therefore better penetration of tissues in vivo,
better safety profile compared to viral delivery vectors with no risk of uncontrolled replication in vivo,
delivery of functional protein to cells with no delay in action which contrasts to DNA delivery in which translation of the DNA into the protein has to occur before an effect,
constructs of the invention are less immunogenic than viral or DNA constructs,
constructs prepared using C3 from Staphylococcus species have the advantage that they are not inhibited by the naturally occurring intracellular protein RalA,
botulinum toxin-targeted C3 rapidly bind to neurons due to their high affinity receptor interaction resulting in efficient action and reduced immune responses, and
possible to specifically target Rho isoforms other than the cytoplasmic RhoA; for example, targeting of the endosomal RhoB has significant therapeutic potential.

A modified clostridial heavy chain for the composition of the invention is suitably produced by combining the binding domain (H_{C} domain) of a clostridial neurotoxin with a non-clostridial translocation domain. Thus, for example, a modified clostridial heavy chain fragment may be constructed from the translocation domain of diphtheria toxin (residues 194-386) fused to the H_{C} domain of a botulinum toxin (e.g. type F H_{C} fragment, residues 865-1278; type A H_{C} fragment, residues 872-1296).

In another embodiment of the invention, the modified clostridial heavy chain is produced by combining the H_{C} domain of a clostridial neurotoxin with a membrane disrupting peptide which functions as a translocation domain, suitably a viral peptide. Thus, for example, a modified clostridial heavy chain fragment may be constructed by combining the H_{C} domain of a botulinum toxin with a peptide based on influenza virus haemagglutinin HA2 (residues 1-23).

In another embodiment of the invention, the modified clostridial heavy chain fragment is fused to a linker peptide via the N-terminus of the translocation domain, to which linker peptide a polypeptide payload may be attached. An example of such a linker peptide is the sequence CGLVPAGSGP (SEQ ID NO:23) which contains the thrombin protease cleavage site and a cysteine residue for disulphide bridge formation. Such a peptide linker allows production of a recombinant fusion protein comprising a polypeptide therapeutic molecule fused by the linker peptide to the N-terminus of the modified clostridial heavy chain fragment. The latter single chain fusion protein may then be treated with thrombin to give a dichain protein in which the polypeptide therapeutic is linked to the translocation domain of the modified clostridial heavy chain fragment by a disulphide link between a free cysteine on the translocation domain and a cysteine residue of the linker peptide. In another example of a linker peptide in which the translocation domain does not contain a free cysteine residue near its C-terminus, such as is the case when the translocation domain is a fusogenic peptide, the linker peptide contains both cysteine residues required for the disulphide bridge. An example of the latter linker peptide is the amino acid sequence: CGLVPAGSGPSAGSSAC (SEQ ID NO:24).

In another embodiment of the invention, the modified clostridial heavy chain is linked to a polypeptide which may be an enzyme, growth factor, protein or peptide which has therapeutic benefits when delivered to neuronal cells. The polypeptide may be linked to the modified clostridial heavy chain by chemical means. Alternatively, the polypeptide may be produced as a fusion protein linked to the modified clostridial binding fragment by recombinant technology using the linker peptides as described above. In such an example, the construct would contain the following components
a polypeptide therapeutic substance;
a linker peptide; and
a modified clostridial heavy chain

In yet another embodiment of the invention, the modified clostridial heavy chain is linked directly or indirectly to DNA such that the construct is capable of delivering the DNA to neuronal cells. Such constructs have gene therapy applications and be used to switch on, or off, selected genes with the cell. The DNA may be contained within a liposome or be condensed via a peptide or protein. The modified clostridial heavy chain may be chemically linked to the protein that effects the DNA condensation by chemical coupling agents. Alternatively, the modified clostridial heavy chain may be produced as a fusion protein, by recombinant technology, with a peptide that can effect the condensation of DNA.

In yet another embodiment of the invention, the modified clostridial heavy chain fragment may be linked to a recombinant virus such that the modified virus has an altered tropism and is capable of transducing cells. Such a construct is of use to correct genetic defects within neuronal cells by switching on, or off, selected genes. The modified clostridial heavy chain fragment may be linked directly to the surface of the virus using chemical cross-linking agents. Alternatively the modified clostridial heavy chain fragment may be linked to the recombinant virus via an antibody which specifically bind to the virus. In this instance the modified clostridial heavy chain fragment is chemically coupled to a polyclonal or monoclonal antibody which specifically recognizes a marker on the surface of the virus. A similar modified clostridial heavy chain fragment-antibody fusion protein could be produced by recombinant technology in which the antibody component is a recombinant single chain antibody.

In yet another embodiment of the invention, the modified clostridial heavy chain fragment is linked to a drug release system such as a microparticle constructed from a suitable polymer, e.g. poly (lactide-co-glycolide), polyhydroxylalkonate, collagen, poly(divinyl-ether-comaleic anhydride, poly (styrene-co-maleic anhydride) or other polymer useful in such microparticles. The modified clostridial heavy chain fragment may be linked to the drug release system by covalent chemical coupling, or electrostatic or hydrophobic forces. The modified clostridial heavy chain fragment may also be encapsulated within the release vehicle together with the therapeutic payload provided that a portion of the modified clostridial binding domain is exposed at the surface. Alternatively, the modified clostridial heavy chain fragment may be linked, at either the N- or C-terminal end, to a peptide or protein to facilitate coupling of the fragment to the drug release system.

Other strategies are known by which modified clostridial heavy chain fragments can be linked to range of therapeutic substances using a variety of established chemical cross-linking techniques, and a variety of fusion proteins can be produced containing a modified clostridial binding fragment and another polypeptide. Using these techniques a variety of substances can be targeted to neuronal cells using the modified clostridial heavy chain fragments. Examples of possible uses of the modified clostridial heavy chain fragments as neuronal delivery vectors are given in more detail below in Constructs of the invention may be introduced into either neuronal or non-neuronal tissue using methods known in the art. By subsequent specific binding to neuronal cell tissue, the targeted construct exerts its therapeutic effects. Ideally, the construct is injected near a site requiring therapeutic intervention.

The construct of the invention may be produced as a suspension, emulsion, solution or as a freeze dried powder depending on the application and properties of the therapeutic substance. The construct of the invention may be resuspended or diluted in a variety of pharmaceutically acceptable liquids depending on the application.

“Clostridial neurotoxin” means either tetanus neurotoxin or one of the seven botulinum neurotoxins, the latter being designated as serotypes A, B C₁, D, E, F or G.

“Modified clostridial heavy chain fragment” means a polypeptide fragment which binds to neuronal cell receptors in similar manner to a corresponding heavy chain derived from botulinum or tetanus toxins but differs in its amino acid sequence and properties compared to the corresponding fragment derived from botulinum or tetanus toxin.

“Bind” in relation to the botulinum and tetanus heavy chain fragments, means the specific interaction between the clostridial fragment and one or more cell surface receptors or markers which results in localization of the binding fragment on the cell surface. In the case of the clostridial neurotoxins, the property of a fragment being able to ‘bind’ like a fragment of a given serotype can be demonstrated by competition between the ligand and the native toxin for its neuronal cell receptor.

“High affinity binding specific to neuronal cell corresponding to that of a clostridial neurotoxin” refers to the ability of a ligand to bind strongly to cell surface receptors of neuronal cells that are involved in specific binding of a given neurotoxin. The capacity of a given ligand to bind strongly to these cell surface receptors may be assessed using conventional competitive binding assays. In such assays radiolabelled clostridial neurotoxin is contacted with neuronal cells in the presence of various concentrations of non-radiolabelled ligands. The ligand mixture is incubated with the cells, at low temperature (0-3° C.) to prevent ligand internalization, during which competition between the radiolabelled clostridial neurotoxin and non-labelled ligand may occur. In such assays when the unlabelled ligand used is the same as that of the labelled neurotoxin, the radiolabelled clostridial neurotoxin will be displaced from the neuronal cell receptors as the concentration of non-labelled neurotoxin is increased. The competition curve obtained in this case will therefore be representative of the behaviour of a ligand which shows “high affinity binding specificity to neuronal cells corresponding to that of a clostridial neurotoxin”, as used herein.

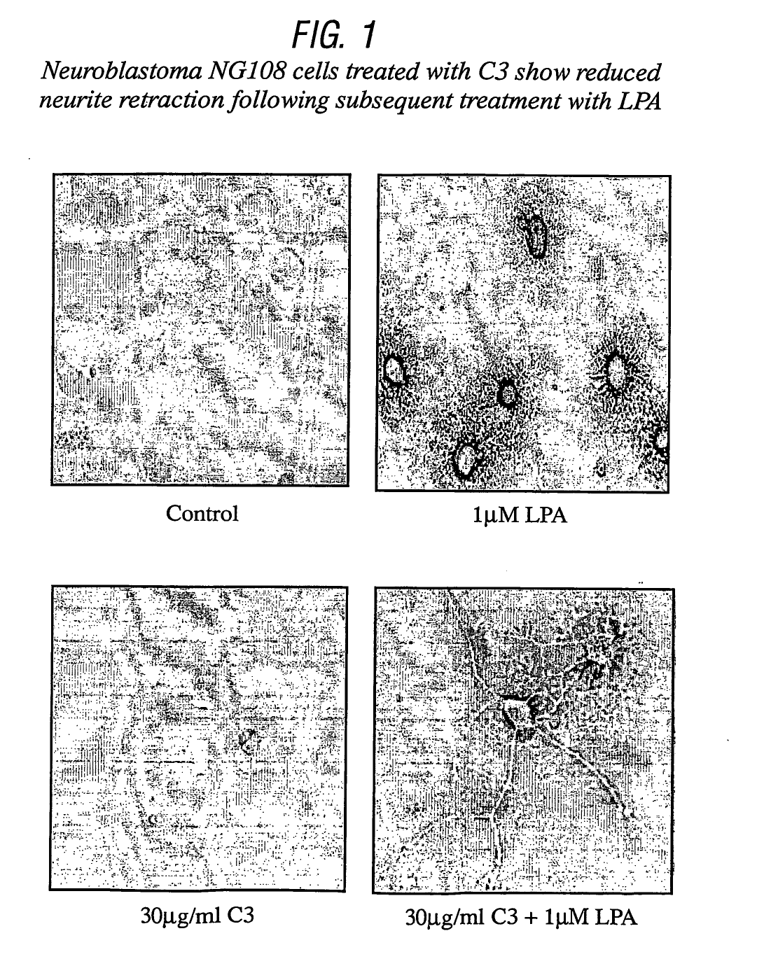

“Translocation domain” means a domain or fragment of a protein which effects transport of itself and/or other proteins and substances across a membrane or lipid bilayer. The latter membrane may be that of an endosome where translocation will occur during the process of receptor-mediated endocytosis. Translocation domains can frequently be identified by the property of being able to form measurable pores in lipid membranes at low pH (Shone et al. Eur J. Biochem. 167, 175-180). Examples of translocation domains are set out in more detail below in FIG. 1. In the application, translocation domains are frequently referred to as “H_{N} domains”.

“Translocation” in relation to translocation domain, means the internalization events which occur after binding to the cell surface. These events lead to the transport of substances into the cytosol of neuronal cells.

“Therapeutic substances” or “agents” mean any substance, agent or mixture thereof, which, if delivered by the modified clostridial binding fragment, would be beneficial to the treatment of neuronal diseases. Examples of these include drugs, growth factors, enzymes, and DNA packaged in various forms (e.g. modified viruses, cationic liposomes, and condensed DNA).

“C3-like activity” means that an enzyme expresses an ADP-ribosyl transferase activity which is specific for small cellular GTPases of the Rho family. In this activity, the C3-like enzyme catalyses the transfer of an ADP-ribose moiety from NAD to the Rho GTPase which usually results in an loss of function of the GTPase.

Also provided in the present invention are methods of manufacture of the polypeptides of the invention by expressing in a host cell a nucleic acid encoding the polypeptide, and the use of a polypeptide or a composition according to the invention in the treatment of a disease state associated with neuronal cells.

The invention is now illustrated in the following specific embodiments and accompanied by drawings in which

FIG. 1 shows protection against neurite reaction in LPA-treated neuroblastoma cells by C3 enzyme; and

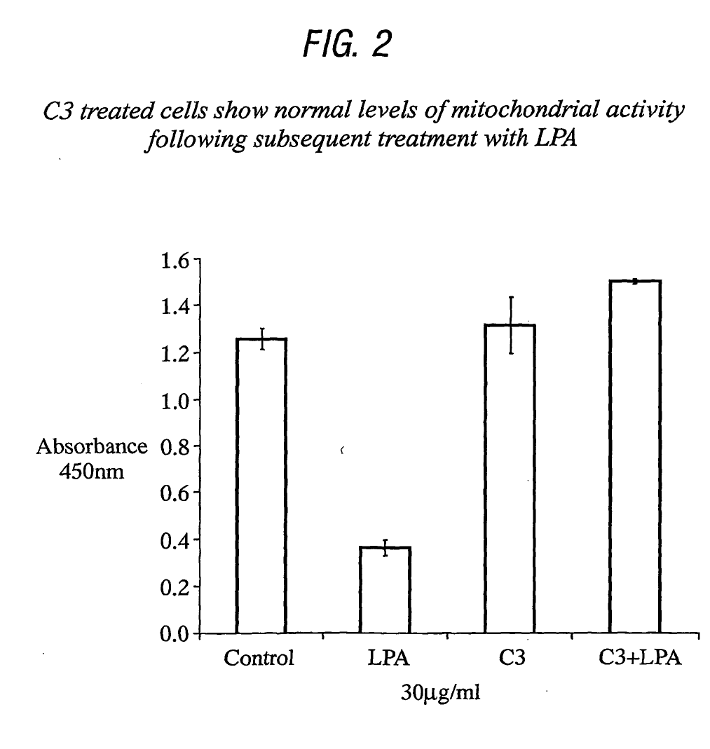

FIG. 2 shows protective or neurostimulatory effect of C3 on LPA-treated neurons.

### EXAMPLES

1. Measurement of Exoenzyme C3 Activity Within an Enzyme

To carry out an assay for the exoenzyme C3, an assay mix was prepared containing the following components
10 μl of Assay Buffer (0.05M Hepes pH 7.2 containing MgCl₂ to a final concentration of 2 mM)
20 μl of recombinant Rho A solution in Assay Buffer (to give 0.5 μg per 100 μl assay)
20 μl of [³²P] NAD⁺ solution in Assay Buffer (to give 1 μCi per 100 μl assay). The NAD being labelled in the alpha phosphate position.

This mixture was incubated at 37° C. for 5 min then 50 μl of C3 enzyme solution (prewarmed to 37° C.) added to start the enzymic reaction.

After incubation for various times at 37° C. (typically between 15-120 min), 100 μl of BSA (2 mg/ml) was added to each assay mix followed by 0.5 ml of 24% trichloroacetic acid solution (TCA) to stop the reaction. The resulting precipitate was centrifuged at high speed on a microfuge for 2 mins, the supernatant fluid decanted and then the precipitate washed with a further 1 ml of 12% TCA solution. The precipitate was resuspended in 1 ml of PBS, by passaging several times through a pipette tip, and then 5 ml scintillation fluid added, mixed and counted in scintillation counter.

The presence of exoenzyme C3, or an enzyme with exoenzyme C3-like activity, was confirmed by the presence of significantly higher radioactivity in the pellet compared to control incubations containing no C3 enzyme.

2. Cloning and Expression of C3 Genes.

Standard molecular biology protocols were used for all genetic manipulations (Sambrook et al 1989, Molecular cloning; A laboratory manual. Second Edition, Cold Spring Harbor Laboratory Press, New York.). C3 genes were amplified by PCR to generate suitable restriction sites for cloning. In some cases synthetic genes were prepared with codon usage optimised for expression in E. coli. The restriction sites BamHI (5′) and BglII (3′) were used for most cloning operations with reading frames designed to start with the first base of the restriction site. Constructs were sequenced to confirm the presence of the correct sequence. An expression vector containing the malE gene from the pMAL-C2x (NEB) subcloned as an ApaI-HindIII fragment into the cloning vector pBC (Strategene) digested ApaI-HindIII was prepared. The C3 genes were cloned into the BamHI site as BamHI-BgIll fragments. Alternatively the C3 genes were cloned into either an expression vector carrying a T7 polymerase promoter site (e.g. pET28, pET30 or derivatives (Novagen Inc, Madison, Wis.)) or as a fusion with maltose binding protein (e.g. pMALc2x (NEB)) as a suitable fragment. Clones with confirmed sequences were used to transform expression hosts. Strain TB1 was used for most expression with alternative hosts used as required (For T7 polymerase vectors E. coli BL21 (DE3) (Studier and Moffatt 1986 Journal of Molecular Biology 189:113-130) JM109 (DE3) or equivalent strains with a DE3 lysogen. For pMalc2x JM109, BL21, TG1, TB1 or other suitable expression strains).

In addition to the expression of C3 proteins as standard fusion proteins an additional approach was used to generate fusions for direct assembly into targeting vectors. C3 fragments were cloned into a BamHI site introduced at the 5′ end of a neuronal cell targeting moiety such as a hybrid diphtheria translocation domain-clostridial neurotoxin binding domain fusion protein

Expression cultures of TB1 pBCmalE C3 were grown in Terrific Broth containing 35 μg/ml chloramphenicol and 0.5% (w/v) glucose to an OD₆₀₀ of 2.0 at 30° C. and cultures were induced with 500 μM IPTG and grown at 25° C. for 2 hours. Other expression systems used similar conditions except that the antibiotic was changed to either kanamycin or ampicillin. Cells were lysed by either sonication in column buffer (20 mM Hepes 100 mM NaCl pH 6.8) or suitable detergent treatment (e.g. Bugbuster reagent; Novagen) and cell debris pelleted by centrifugation. Supernatant proteins were loaded onto a SP-sepharose Fast Flow column equilibrated in column buffer and proteins eluted with a gradient of 0-1 M NaCl. MBP-C3 fusions eluted at 200 mM NaCl. Fusion protein was cleaved with Factor Xa in 20 mM Hepes 200 mM NaCl pH6.8 (as eluted) to separate the C3 domain from its MBP fusion partner. The protein was diluted to give a final buffer concentration of 20 mM Hepes 100 mM NaCl pH 6.8 and loaded onto the SP-Sepharose column under identical conditions to those used initially. C3 protein, essentially free of contaminating proteins, was eluted at ˜400 mM NaCl. Protein was stored at −70° C. until required.

The C3stau2 isoform can be purified using a similar method. Cells are lysed in 20 mM MES 50 mM NaCl pH5.8 and the soluble material loaded on to an SP-sepharose column. The protein elutes at around 150 mM NaCl. The protein is dialysed against 20 mM HEPES 50 mM NaCl pH 7.3 and cleaved with Factor Xa. The protein is passed through a second SP-sepharose column and elutes at approximately 250 mM NaCl. Protein is stable at −70° C.

His-tag fusions were loaded onto a metal chelate column charged with Cu²⁺ (Amersham-Pharmacia Biotech, Uppsala, Sweden). After loading proteins on the column and washing, proteins were eluted using imidazole. All buffers were used as specified by manufacturers. Where appropriate removal of the purification tag was carried out according to manufacturers instructions.

MBP fusions could also be purified on amylose resin columns as described by the manufacturer (NEB) following growth in Terrific Broth containing 100 μg/ml ampicillin and lysis as described above.

3. Expression of Heavy Chain Fragments.

Standard molecular biology protocols were used for all genetic manipulations (Sambrook et al 1989, Molecular cloning; A laboratory manual. Second Edition, Cold Spring Harbor Laboratory Press, New York.) Clostridial neurotoxin binding domains (BoNT/Hc or TeNT/Hc) derived from either their native genes or synthetic genes with codon usage optimised for expression in E. coli were amplified by PCR. Introduced BamHI (5′) restriction sites and HindIII, SalI or EcoRI (3′) sites were used for most cloning operations with reading frames designed to start with the first base of the restriction site. Constructs were sequenced to confirm the presence of the correct sequence. The translocation domain of diphtheria toxin (DipT) was amplified from its native gene to introduce BamHI and BglII sites at the 5′ and 3′ ends respectively. This BamHI and BglII fragment was subcloned into the BamHI site at the 5′ end of the Hc fragment to generate an in-frame fusion. The entire heavy chain fragment (DipT-Hc) was excised as a BamHI-HindIII or BamHI-SalI or BamHI-EcoRI fragment and subcloned into a suitable expression vector.

Constructs for expression were subcloned into either an expression vector carrying a T7 polymerase promoter site (e.g. pET28, pET30 or derivatives (Novagen Inc, Madison, Wis.)) or to generate a fusion with maltose binding protein (e.g. pMALc2x (NEB)) as a suitable fragment. Clones with confirmed sequences were used to transform expression hosts: For T7 polymerase vectors E. coli BL21 (DE3) (Studier and Moffatt 1986 Journal of Molecular Biology 189:113-130) JM109 (DE3) or equivalent strains with a DE3 lysogen. For pMalc2x JM109, BL21, TG1, TB1 or other suitable expression strains.

The recombinant proteins expressed from pET vectors contain amino-terminal histidine (6-His) and T7 peptide tags allowing proteins to be purified by affinity chromatography on either a Cu²⁺ charged metal chelate column. Expression cultures were grown in Terrific Broth containing 30 μg/ml kanamycin and 0.5% (w/v) glucose to an OD₆₀₀ of 2.0 and protein expression was induced with 500 μM IPTG for 2 hours. Cells were lysed by either sonication or suitable detergent treatment (e.g. Bugbuster reagent; Novagen), cell debris pelleted by centrifugation and the supernatant loaded onto a metal chelate column charged with Cu²⁺ (Amersham-Pharmacia Biotech, Uppsala, Sweden). After loading proteins on the column and washing, proteins were eluted using imidazole. All buffers were used as specified by manufacturers. Where appropriate removal of the purification tag was carried out according to manufacturers instructions.

MBP fusions were purified on amylose resin columns as described by the manufacturer (NEB) following growth in Terrific Broth containing 100 μg/ml ampicillin and lysis as described above.

4. Purification of the Heavy Chain of Botulinum Type C₁ Neurotoxin.

Step 1. Botulinum type C₁ neurotoxin (5 mg total) is dialysed against borate/phosphate buffer pH 8.5 (29 mM Na₂B₄O₇; 42 mM NaH₂PO₄ titrated to pH 8.5 with NaOH) and then applied (0.5 ml min⁻¹) to a column (1 cm×5 cm) of QAE-Sephadex (Pharmacia) equilibrated in the borate/phosphate pH 8.5 buffer. The column is then washed with a further 10 ml of buffer followed by 10 ml of the borate/phosphate buffer containing 10 mM dithiothreitol. After the latter buffer has been allowed to run almost completely onto the column, 3 ml of borate/phosphate pH 8.5 buffer containing 100 mM dithiothreitol and 2M urea are carefully applied to the column and 2.5 ml of this buffer allowed to run onto the gel. The column is then sealed and incubated at 4° C. overnight.

Step 2. The light subunit of the neurotoxin is eluted with 20 ml of borate/phosphate pH 8.5 buffer containing 2M urea and 10 mM dithiothreitol collecting 1.5 ml fractions.

Step 3. The heavy subunit of the type C₁ neurotoxin is eluted with 20 ml of borate/phosphate pH 8.5 buffer containing 2M urea, 10 mM dithiothreitol and 0.2M NaCl. The presence of heavy chain in eluted fractions (1 ml) may be detected by uv measurement. Those fractions containing the highest concentration of protein are pooled and then dialysed against 0.05M Hepes pH 7.4 buffer containing 0.15M NaCl. The purified type C, neurotoxin may be stored frozen at −80° C. until required.

5. Preparation of Chemical C3-Heavy Chain Conjugates.

Purified C3 was dialysed into phosphate buffered saline (PBS) pH7.4. Sulfo-LC-SPDP was added to give a final molar excess of 3:1 SPDP:C3 and reacted for 2 hours at room temperature. Free SPDP was removed by dialysis. Derivitised C3 was incubated with either native or recombinant heavy chain fragments containing a free cysteine residue for 20 hours at 4° C. Free C3 could be removed if required by cation exchange chromatography on a MonoS column run under the same conditions as the SP-Sepharose column described in example 1. Additional purification is also possible on a Cibacron-Blue affinity resin if required.

6 Assessment of Activity of C3 Conjugates on Neuroblastoma Cells.

NG108 neuroblastoma cells were cultured in DMEM containing 10% foetal calf serum (FCS) and 2 mM glutamine at 37° C. at 5% CO₂ saturation. Cells were plated onto poly-D-lysine coated 96 well culture plates at a density of 2×10⁴. After 24 hours the cells were changed into DMEM/glutamine medium lacking FCS and grown for a further 16 hours as above. Conjugate or free C3 was added and the cells incubated for either 3 hours or overnight. The cells were treated with 1 μM lyso-phosphatidic acid (LPA) in DMEM/glutamine. Cells were observed for morphological changes under a microscope. As shown in FIG. 1 C3 at a final concentration of 30 μg/ml protected the cells from LPA mediated neurite retraction. To assay for cellular events in response to LPA treatment an MTT assay was used as described previously (Alley M C, Scudiero D A, Monks A, Hursey M L, Czerwinski M J et al (1988) Feasibility of drug screening with panels of human tumor cell lines using a microculture tetrazolium assay. Cancer Res 48, 589-601). Assay results shown in FIG. 2 show a marked reduction in mitochondrial activity, as measured by reduction of MTT, in LPA treated cells. Cells pre-treated with C3 (30 μg/ml final concentration) before addition of LPA at showed similar levels of mitochondrial activity to untreated control cells indicating that C3 prevents LPA-induced cellular damage. This is indicative of protective or neurostimulatory effect in damaged neurons.

Expressed Sequence Identifications.

In the sequence listing accompanying this specification, the sequences have the following derivation

Sequence 1 C3 protein from Clostridium botulinum lacking the N-terminal signal sequence and including a factor Xa cleavge site immediately adjacent to the initial Alanine amino acid of the mature sequence.

Sequence 2 C3 protein from Staphylococcus aureus lacking the N-terminal signal sequence and including a factor Xa cleavage site immediately adjacent to the initial Alanine amino acid of the mature sequence. The protein refers to the C3Stau 2 isoform sometimes referred to as EDIN B

Sequence 3 Gene encoding C3 protein from Staphylococcus aureus lacking the N-terminal signal sequence and including a factor Xa cleavage site immediately adjacent to the initial Alanine amino acid of the mature sequence. The synthetic gene, with codon usage optimised for expression in E. coli, encodes the C3Stau 2 isoform sometimes referred to as EDIN B

Sequence 4 mature C3 protein from Staphylococcus aureus; termed C3Stau 1 (EDIN A)

Sequence 5 full length C3 protein from Staphylococcus aureus; termed C3Stau 1 (EDIN A) including a signal sequence at the N-terminus.

Sequence 6 mature C3 protein from Clostridium limosum

Sequence 7 C3-like protein from Listeria monocytogenes

Sequence 8 C3-like protein from Clostridium acetobutylicum

Sequence 9 C3-like protein from Streptococcus pyogenes (native protein sequence)

Sequence 10 Second C3-like protein from Streptococcus pyogenes (native protein sequence)

Sequence 11 shows the native heavy chain (HC) of BoNT/C1 with the di-chain linker region. The native Factor Xa cleavage site within the linker region is also present. Optionally the fragment can also include the amino acids GS at the N-terminus, encoded by an in-frame BamHI site used for cloning purposes.

Sequence 12 shows the isolated receptor binding domain H_{C} from BoNT/C1. This corresponds to the C-terminal domain of the H_{C} and has been shown to confer the ability to bind to neuronal receptors. Optionally the domain sequence is preceded at the N-terminus by the amino acids GS encoded for by an in-frame BamHI site used for cloning of the fragment.

Sequence 13 In expression constructs the first alanine residue shown is preceded by a factor Xa cleavage site such that the proteolytic processing removes all of the fusion partner. The intrachain linker is simultaneously cleaved during the same processing step.

Sequence 14 shows the fusion protein C3Stau2 BoNT/C1-HC with the di-chain linker region. In expression constructs the first alanine residue shown is preceded by a factor Xa cleavage site such that the proteolytic processing removes all of the fusion partner. The intrachain linker is simultaneously cleaved during the same processing step.

Sequence 15 shows the fusion protein C3bot BoNT/C1-H_{C} with the linker region derived from the native BoNT/C1. In expression constructs the first alanine residue shown is preceded by a factor Xa cleavage site such that the proteolytic processing removes all of the fusion partner. The intrachain linker is simultaneously cleaved during the same processing step. This construct differs from Sequence 13 in that it does not contain the H_{N} domain and as such lacks a translocation function. This alters the intracellular trafficking of the C3 fragment in cells. Optionally the linker can be removed, with the C3 fragment joined directly onto the BoNT/C1-H_{C} fragment. The GS amino acid residues following the linker sequence are derived from the BamHI site used in the construction of the fragment.

Sequences 16-24 show a variety of alternative linkers suitable for joining the C3 entity to the BoNT/C1 HC or H_{C} fragment. In variant embodiments, these linkers optionally replace the native linker sequence, identified as Sequence 22. In several instances these linkers can be cleaved by a different specific proteases allowing the sequential processing of the fusion site with Factor Xa and the intra-chain site.

1-50. (canceled)

51. A composition, for delivery of a therapeutic agent to a neuronal cell, comprising:
a therapeutic agent which inhibits at least one member of the Rho group of GTPases, and
a neuronal cell targeting component, which component comprises a Hc domain of botulinum C1 toxin, or a fragment thereof which retains the function of the native Hc domain,
wherein the Hc domain has been made recombinantly.

52. A composition according to claim 51 further comprising a domain for translocation of the therapeutic agent into a cell.

53. A composition according to claim 52 wherein the translocation domain is derived from a clostridial source.

54. A composition according to claim 52 wherein the translocation domain is derived from a non-clostridial source.

55. A composition according to claim 53 wherein the translocation domain is derived from C. botulinum, C. butylicum, C. argentinense or C. tetani.

56. A composition according to claim 54 wherein the translocation domain comprises a translocation domain of diphtheria toxin, Pseudomonas exotoxin A, influenza virus haemagglutinin fusogenic peptides or amphiphilic peptides.

57. A composition according to claim 52, wherein the translocation domain comprises a member selected from the group consisting of botulinum C1 toxin and fragments thereof, and diphtheria toxin and fragments thereof.

58. A composition according to claim 52 wherein the translocation domain is a membrane disrupting peptide.

59. A composition according claim 51, wherein the therapeutic agent is selected from the group consisting of drugs, growth factors, enzymes, DNA, modified viruses, drug release systems, and a combination thereof.

60. A composition according to claim 51, wherein the therapeutic agent is a C3 enzyme.

61. A composition according to claim 60, wherein the C3 enzyme is derived from C. botulinum, C. limosum, B. cereus, S. aureus, C. acetobutylicum, S. pyogenes, L. monocytogenes.

62. A composition according to claim 60, wherein the C3 enzyme is selected from the group consisting of C3Stau2, C3Stau1, and C3bot.

63. A composition according to claim 60, wherein the C3 enzyme has an amino acid sequence selected from the group consisting of SEQ ID Nos: 1-10.

64. A composition according to claim 51, wherein the therapeutic agent and the Hc domain are joined to each other directly or via a linker molecule.

65. A composition according to claim 52, wherein the therapeutic agent, the Hc domain and the translocation domain are joined to each other directly or via a linker molecule.

66. A composition according to claim 64, wherein the linker molecule is selected from the group consisting of (GGGGS)2, (GGGGS)3, the interdomain linker of cellulase, PPPIEGR, collagen-like spacer, trypsin-sensitive diphtheria toxin peptide, and linker molecules having an amino acid sequence of SEQ ID Nos: 16-24.

67. A composition according to claim 65, wherein the linker molecule is selected from the group consisting of (GGGGS)2, (GGGGS)3, the interdomain linker of cellulase, PPPIEGR, collagen-like spacer, trypsin-sensitive diphtheria toxin peptide, and linker molecules having an amino acid sequence of SEQ ID Nos: 16-24.

68. A composition according to claim 51, wherein the composition is a single polypeptide.

69. A composition according to claim 51, wherein the composition is a dichain polypeptide.

70. A composition according to claim 51, wherein the composition is a suspension, emulsion, solution or a freeze-dried powder.

71. A composition according to claim 51, further comprising a pharmaceutically acceptable liquid.

72. A method of making a composition according to claim 51, comprising expressing a DNA encoding the therapeutic agent and the neuronal cell targeting domain.


SEQUENCE LISTING (24 sequences)

SEQ ID NO: 1 - protein, 215 aa, Artificial Sequence
  Synthetic
IEGRAYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPK

SEQ ID NO: 2 - protein, 212 aa, Artificial Sequence
  Synthetic
AETKNFTDLVEATKWGNSLIKSAKYSSKDKMAIYNYTKNSSPINTPLRSANGDVNKLSENIQEQVRQLDSTISKSVTPDSVYVYRLLNLDYLSSITGFTREDLHMLQQTNNGQYNEALVSKLNNLMNSRIYRENGYSSTQLVSGAALAGRPIELKLELPKGTKAAYIDSKELTAYPGQQEVLLPRGTEYAVGSVKLSDNKRKIIITAVVFKK

SEQ ID NO: 3 - protein, 636 aa, Artificial Sequence
  Synthetic
GCTGAAACCAAAAACTTCACCGACCTGGTTGAAGCTACCAAATGGGGTAACTCTCTGATCAAATCTGCTAAATACTCTTCTAAAGACAAAATGGCTATCTACAACTACACCAAAAACTCTTCTCCGATCAACACCCCGCTGCGTTCTGCTAACGGTGACGTTAACAAACTGTCTGAAAACATCCAGGAACAGGTTCGTCAGCTGGACTCTACCATCTCTAAATCTGTTACCCCGGACTCTGTTTACGTTTACCGTCTGCTGAACCTGGACTACCTGTCTTCTATCACCGGTTTCACCCGTGAAGACCTGCACATGCTGCAGCAGACCAACAACGGTCAGTACAACGAAGCTCTGGTTTCTAAACTGAACAACCTGATGAACTCTCGTATCTACCGTGAAAACGGTTACTCTTCTACCCAGCTGGTTTCTGGTGCTGCTCTGGCTGGTCGTCCGATCGAACTGAAACTGGAACTGCCGAAAGGTACCAAAGCTGCTTACATCGACTCTAAAGAACTGACCGCTTACCCCGGTCAGCAGGAAGTTCTGCTGCCGCGTGGTACCGAATACGCTGTTGGTTCTGTTAAACTGTCTGACAACAAACGTAAAATCATCATCACCGCTGTTGTTTTCAAGAAG

SEQ ID NO: 4 - protein, 212 aa, Staphylococcus aureus
ADVKNFTDLDEATKWGNKLIKQAKYSSDDKIALYEYTKDSSKINGPLRLAGGDINKLDSTTQDKVRRLDSSISKSTTPESVYVYRLLNLDYLTSIVGFTNEDLYKLQQTNNGQYDENLVRKLNNVMNSRIYREDGYSSTQLVSGAAVGGRPIELRLELPKGTKAAYLNSKDLTAYYGQQEVLLPRGTEYAVGSVELSNDKKKIIITAIVFKK

SEQ ID NO: 5 - protein, 247 aa, Staphylococcus aureus
MKRKLFFKIIFVLSLVLSIHSINDRTTELSNIALADDVKNFTDLTEATNWGNKLIKQANYSSKDKEAIYNYTKYSSPINTPLRSSQGDISNFSADLQEKILRLDRLISKSSTSDSVYVYRLLNLDYLSSVKGFSSEDLELLYKTENGKYNEELVKKLNNIMNSKIYTEYGYSSTQLVKGAALAGRPIELKLQLPKGTKAAYIDSKNLTAYPGQQEILLPRGTDYTINTVKLSDDHKRILIEGIVFKK

SEQ ID NO: 6 - protein, 211 aa, Clostridium limosum
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPK

SEQ ID NO: 7 - protein, 160 aa, Listeria monocytogenes
NKSLKFTSLEESEKWGIDGFSVWRNSLSSREIQAIRDYTDIWHYGNMNGYLRGSVEKLAPDNAERIKNLSSALEKAELPDNIILYRGTSSEILDNFLDLKNLNYQNLVGKTIEEKGFMSTTTISNQTFSGNVTMKINAPKGSKGAYLAHFSETPEEAEVL

SEQ ID NO: 8 - protein, 175 aa, Clostridium acetobutylicum
TNMDQANEWGSQYYDNWLKSLNDSERNAIRQYTGNDYKKINNYLRGVNDSLDGIDPKIIEDIKSGLKKASVPHDMKVYRGTDLNPLRNLIDVGKDGSLDFSLVGKTFKDDGFMSTALVKESSFDYMNVSWEINVPKGTEAAYVSKISYFPDEAELLLNHGQEMIIKEATVGSDGK

SEQ ID NO: 9 - protein, 250 aa, Streptococcus pyogenes
MLKKRYQLAIVLLLSCFSLIWQTEGLVELFVCEHYERAVCEGTPAYFTFSDQKGAETLIKKRWGKGLIYPRAEQEAMAAYTCQQAGPINTSLDKAKGELSQLTPELRDQVAQLDAATHRLVIPWNIVVYRYVYETFLRDIGVSHADLTSYYRNHQFDPHILCKIKLGTRYTKHSFMSTTALKNGAMTHRPVEVRICVKKGAKAAFVEPYSAVPSEVELLFPRGCQLEVVGAYVSQDQKKLHIEAYFKGSL

SEQ ID NO: 10 - protein, 250 aa, Streptococcus pyogenes; identical to SEQ ID NO: 9

SEQ ID NO: 11 - protein, 855 aa, Clostridium botulinum
CHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 12 - protein, 454 aa, Clostridium botulinum
GSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 13 - protein, 1066 aa, Artificial Sequence
  Synthetic
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPKCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 14 - protein, 1067 aa, Artificial Sequence
  Synthetic
AETKNFTDLVEATKWGNSLIKSAKYSSKDKMAIYNYTKNSSPINTPLRSANGDVNKLSENIQEQVRQLDSTISKSVTPDSVYVYRLLNLDYLSSITGFTREDLHMLQQTNNGQYNEALVSKLNNLMNSRIYRENGYSSTQLVSGAALAGRPIELKLELPKGTKAAYIDSKELTAYPGQQEVLLPRGTEYAVGSVKLSDNKRKIIITAVVFKKCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 15 - protein, 682 aa, Artificial Sequence
  Synthetic
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPKCHKAIDGRSLYNKTLDCGSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 16 - protein, 12 aa, Artificial Sequence
  Synthetic
CSAIEGRAPGIC

SEQ ID NO: 17 - protein, 20 aa, Artificial Sequence
  Synthetic
CGIEGRAPGPGSSVGSSLSC

SEQ ID NO: 18 - protein, 11 aa, Artificial Sequence
  Synthetic
CGLVPRGSGPC

SEQ ID NO: 19 - protein, 20 aa, Artificial Sequence
  Synthetic
CGLVPRGSGPGSSVGSSLSC

SEQ ID NO: 20 - protein, 13 aa, Artificial Sequence
  Synthetic
CKSDDDDKAPGIC

SEQ ID NO: 21 - protein, 22 aa, Artificial Sequence
  Synthetic
CKSEEKLYDDDDKDRWGSSRIC

SEQ ID NO: 22 - protein, 17 aa, Clostridium Botulinum
CHKAIDGRSLYNKTLDC

SEQ ID NO: 23 - protein, 10 aa, Artificial Sequence
  Synthetic
CGLVPAGSGP

SEQ ID NO: 24 - protein, 17 aa, Artificial Sequence
  Synthetic
CGLVPAGSGPSAGSSAC